(19) United States
(12) Patent Application Publication
(10) Pub. No.: US 2012/0012669 A1
(43) Pub. Date: Jan. 19, 2012

(54) Fan-mounted air freshener device and associated method

(76) Inventor: Jamal McBride, Neptune, NJ (US)

(21) Appl. No.: 13/135,013

(22) Filed: Jun. 23, 2011

Related U.S. Application Data
(60) Provisional application No. 61/365,002, filed on Jul. 16, 2010.

(51) Int. Cl. A61L 9/04

(52) U.S. Cl. 239/34

(57) ABSTRACT

A fan-mounted air freshener device provides users with an easy and convenient means of emitting bursts of air-freshening scents or deodorizing sprays into a room. A control panel comprises a plurality of control knobs for selecting a variety of scents or sprays, and a timer for setting a scent-release interval. Bursts of scent or spray are thereby emitted during operation of the fan, as desired by the user. The fan-blades comprise conduits that terminate at a nozzle, which is located near the top of the fan blades. The scent or spray is conveyed to the fan-blades via conduits or canals, which originates at the scent reservoir. The scent or spray continues to flow through the conduits and is emitted into the ambient air via the nozzle. Centrifugal forces created by the whirling fan blades fling droplets of scent or spray into the room in a rotary array via the nozzles.

### BACKGROUND OF THE INVENTION

This Application claims the benefit of provisional application No. 61/365,002, filed Jul. 16, 2010.

1. Field of the Invention

This invention relates to fans; and more particularly, to a fan-mounted air freshener device for providing users with an easy and convenient means of emitting bursts of air-freshening scents or deodorizing sprays in a room via the sweep of air from the fan blades.

2. Description of the Prior Art

Modern day electric fans—including both ceiling fans and table fans—have a lineage that extends back to the 16th century “punkah” fans of South Asia, which were operated manually. During the European and American Industrial Revolutions in the early 1800s, large belt-driven fans powered by factory water wheels were introduced. In 1882, an inventor named Philip H. Diehl introduced the electric rotary ceiling fan. In Diehl's time, electric fans were used only in commercial establishments or in well-to-do households. In our own day, however, electric fans are commonplace.

Typical examples of electric fans, as noted, are ceiling fans and table fans. A ceiling fan is attached to and suspended from the ceiling of a room; it is designed to move air through the whole room. A table fan, by contrast, is portable and typically smaller than a ceiling fan; it is designed to rest on a table, countertop or desk. A table fan frequently functions as a type of axial-flow fan, which is designed to force the air to move parallel to the axis about which its blades rotate; said axis is aligned to be substantially parallel to the room's floor. Handheld electric fans that are usually small and battery powered are also available for use.

The key components of an electric fan include an electric motor, which is powered by a power source such as an AC outlet or battery. The electric motor spins a motor shaft, to which blades (or vanes) are attached. It is this rotation of the blades about the motor shaft that acts on the air, thereby forcing the air to move.

Electric fans are typically used in settings that lack central air conditioning, or in conjunction with air conditioning to reduce energy bills. Electric fans can be used as a cooling device in warm months, pushing air to create a wind-chill effect; and as a heat transferor during colder months, pulling in warm air. Electric fans may further be used for providing ventilation, fume extraction, dust removal, drying and to provide a draft for a fire.

While electric fans are an effective and inexpensive means of moving air to accomplish the above noted tasks, they do nothing to purify or freshener the air they move. The art has failed to produce an invention capable of increasing the utility of electric fans by adding an air-freshening or deodorizing quality to their functions. Accordingly, a need remains for an apparatus in order to overcome these shortcomings.

The present invention satisfies such a need by providing a fan-mounted air freshener device that is convenient and easy to use, lightweight yet durable in design, versatile in its applications, and designed for emitting bursts of air-freshening scents or deodorizing sprays into a room via the sweep of air from the fan blades.

### SUMMARY OF THE INVENTION

The present invention provides a fan-mounted air freshener device capable of being used to emit bursts of air-freshening scents or deodorizing sprays—in either liquid or vaporized form—into a room. The scent or spray travels along the length of the fan-blades via a tube-like conduit, and these scents or sprays are dispensed and released into the ambient air from a nozzle that is preferably positioned near the tips of the blades. As the fan's blades rotate, the centrifugal forces they create push the scent droplets or vapor out in a rotary array, thereby spreading the air-freshening scents or deodorizing sprays throughout the room while simultaneously moving air.

Almost all types of fans are capable of being used for this invention, but household fans, such as ceiling fans and table fans, appear to be the best suited for effectuating the invention's intended air freshening purpose. Fans to be used can be selected from a wide variety of styles, colors, finishes and price ranges. In each case, however, the distinctive aspect of this invention would be its air freshening function, as noted above.

The fan-mounted air freshener device comprises a control panel or control unit, allowing the user to determine when the scent or spray is to be released. In preferred embodiments of the invention, the controls may be used to operate a timer capable of being programmed to release the scent or spray at any predetermined time interval desired. The controls may further offer the user a variety of scents or sprays to choose from. The ceiling fan embodiment of this invention is preferably equipped with a wall-mounted control panel. A remote control may also be used to operate the device.

The scent or spray is stored in a scent reservoir, which comprises one or more storage tanks capable of releasing emissions of said scent or spray when directed to do so. Thus, when directed by the user (via operation of the controls), said scent reservoir releases emissions of the scent or spray, which are subsequently conveyed to the conduits of the fan-blades by way of a tube-like canal. Almost any variety of air-freshening scent or deodorizing spray can be used with this invention. The user may refill the reservoir to replace the depleted scent or spray when needed. Scents or spray may be supplied by replaceable cartridges, which are offered in different varieties. In other embodiments, the scent or spray may be replaced by simply pouring a liquid directly into the reservoir. As noted, the scent reservoir may comprise multiple tanks, thus allowing it to separately store different varieties of scents and/or sprays.

Although the preferred embodiments of the fan-mounted air freshener device comprise modified fans (whose blades are constructed to comprise the above noted nozzles and conduits), this invention is also available as an aftermarket accessory item. Accordingly, the fan-mounted air freshener apparatus can be easily installed on any existing table or ceiling fan, thereby retrofitting the existing fan so that it can have air freshening capabilities. This embodiment would comprise peel-n-stick adhesive-backed components, which the user would affix onto the fan's blades. For example, peel-n-stick adhesive-backed air-freshener packets could be affixed to the blades of an existing fan, thereby providing air-freshening qualities for a limited time period. These air-freshener packets are disposable and can be purchased in multiunit packages.

As has become apparent, the fan-mounted air freshener device may embody many different forms. Accordingly, the invention should not be construed as being limited to those embodiments set forth herein. Rather, these embodiments are provided so that the true scope of this invention will be conveyed to those skilled in the art. Further, this disclosure should not be construed as limiting use of the fan-mounted air freshener device to only those uses described herein.

Advantageously, the fan-mounted air freshener device provides a ceiling or table fan that is capable of both ventilating a room and dispersing aromatic scents at preset time intervals. This invention would effectively and automatically freshen the air of the room in which it was employed, while presenting the user with a wide variety of options in terms of style, model, scent and price.

### BRIEF DESCRIPTION OF DRAWINGS

The invention will be more fully understood and further advantages will become apparent when reference is made to the following detailed description of the preferred embodiments of the invention and the accompanying drawings, in which:

FIG. 1 depicts a fan-mounted air freshener device, wherein the fan is a ceiling fan; and

FIG. 2 depicts a fan-mounted air freshener device, wherein said fan is a table fan.

### DETAILED DESCRIPTIONS OF THE PREFERRED EMBODIMENTS

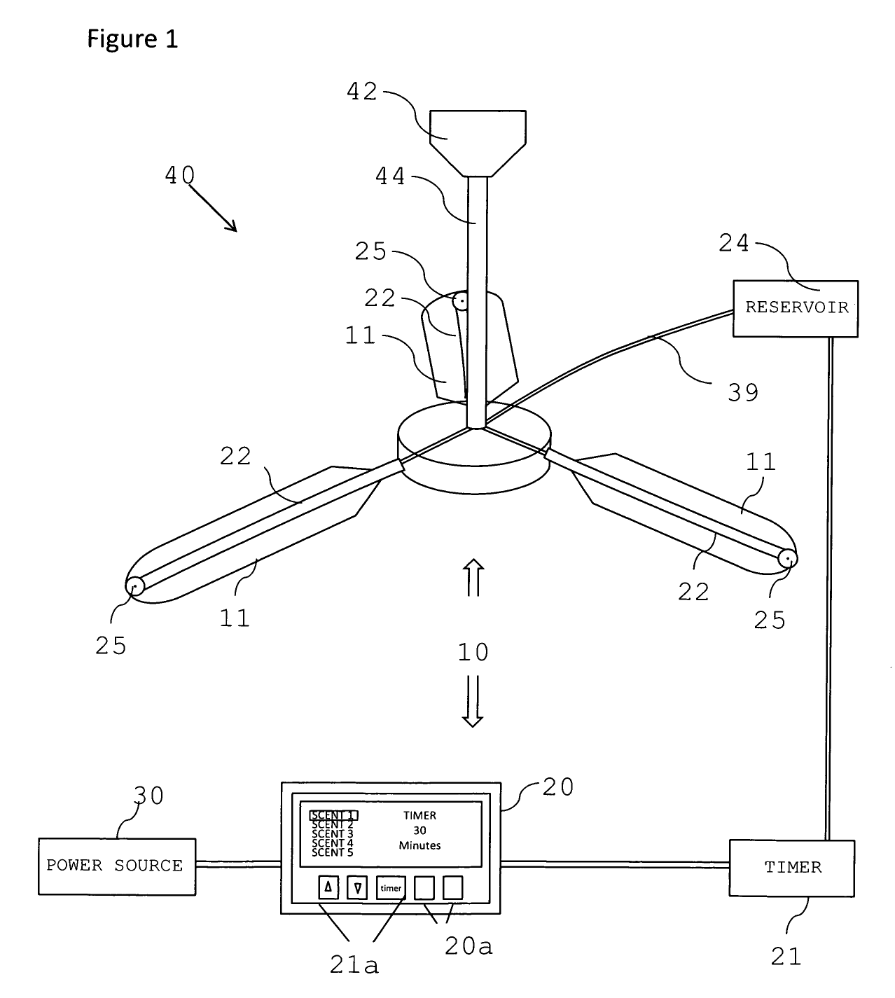

Reference is made to FIG. 1, which depicts the fan-mounted air freshener device 10 as being mounted on a ceiling fan 40. The invention comprises a wall-mounted control panel 20 with buttons 20a for selecting an air-freshening scent or deodorizing spray from a variety of options that are available. The control panel 20 further includes timer control buttons 21a, which programs a timer 21 in order to set intervals for release of the selected scent or spray. The control panel 20 is connected to a power supply source 30, such as a household AC outlet. Ceiling fan 40 comprises all the salient features of a conventional electric fan, including the electric motor 42 and the motor shaft 44.

Each fan blade 11 is comprised of a conduit 22 such that a selected scent or spray may flow through the conduit 22 at the command of the control panel 20. This conduit 22 terminates at a nozzle 25, which is positioned near the top ends of the fan blades 11. In this way, centrifugal force created by the whirling fan blades 11 flings the scent droplets or vapor out into the room in a rotary array, once they are released into the ambient air from the nozzles 25. The conduits 22 may be located on either the upper or lower side of the fan-blades 11, or they may be constructed within the actual body of the fan-blades 11.

The scent is conveyed to the fan-blades 11 by way of a conduit or canal 39, which originates at the scent reservoir 24. In essence, the canal 39 differs from the conduits 22 only in that it (the canal) is not located on the fan-blades 11; otherwise, both the canal 39 and the conduits 22 function in the same manor. These components serve as a means through which the scent or spray travel, from the reservoir 24 to the nozzle 25. The canal 39 and conduits 22 are both tube-like structures, the diameter of the canal 39 typically being greater than that of the conduits 22.

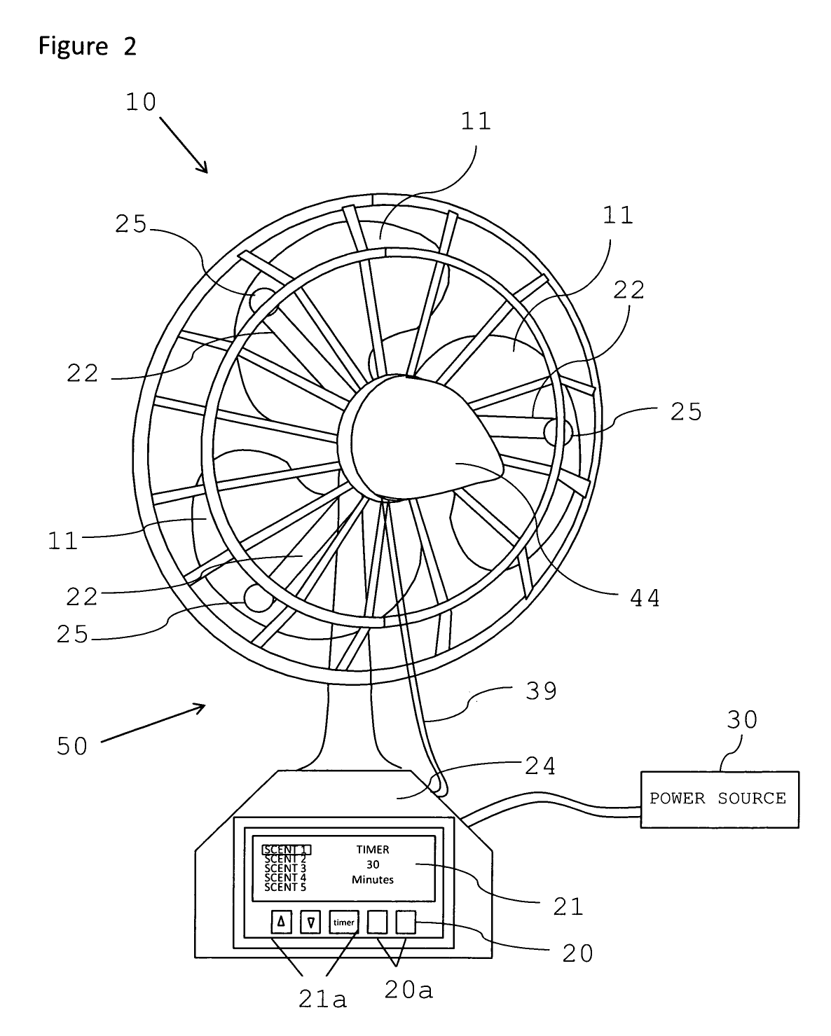

Referring to FIG. 2 of the drawings, there is shown a fan-mounted air freshener device 10 mounted on a table fan 50. The fan-mounted air freshener device 10 comprises a control panel 20 with buttons 20a for selecting an air-freshening scent or deodorizing spray from the options that are available. The control panel 20 further includes timer control buttons 21a, which programs a timer 21 in order to set intervals for release of the selected scent or spray. The table fan 50 is connected to a power supply source 30, such as a household AC outlet. Each fan blade 11 comprises a conduit 22 that terminates at a nozzle 25; said nozzle 25 is positioned near the top ends of the fan blades 11. The scent is conveyed to the fan-blades 11 by way of the canal 39, which originates at the scent reservoir 24. In this embodiment, it is preferable for the conduits 22 to be positioned on the front side of the fan blades 11, so that the centrifugal force created by the revolving fan blades 11 flings the scent droplets or vapor forward and into the ambient air.

Generally stated, the embodiment of the fan-mounted air freshener device 10 that is mounted on a table fan 50 comprises the same features as the embodiment that is mounted on a ceiling fan 40. The primary difference being that the table fan embodiment is smaller, lighter and more compact than the ceiling fan embodiment, thus making this table fan embodiment portable.

In an alternative embodiment, the fan-mounted air freshener device 10 is an aftermarket accessory that may be installed on an existing table, ceiling or other electric fan. This embodiment does not itself comprise an electric fan, but rather it consists of an apparatus that is used to retrofit an existing electric fan, such that said fan acquires air freshening capabilities. This embodiment comprises conduits 22 having a connection means on a portion of their outer surface that is appointed to attach said conduits 22 to the fan-blades 11. This connection means may be, for example, a thin layer of an adhesive or a peel-n-stick adhesive-backed component. A user can easily attach said conduits to the fan-blades 11 of an existing electric fan, thereby adapting the fan-blades 11 so that they comprise means through which the scent or spray travels to the nozzle 25; said nozzle 25 is merely the opening at the end of the conduits 22. In addition to the conduits 22, this embodiment of the fan-mounted air freshener device 10 also comprises controls 20, a timer 21, a scent reservoir 24 and canals 39. Therefore, once the conduits 22 are attached to the fan-blades 11, the apparatus together with the electric fan substantially resembles the embodiments depicted in FIG. 1 and FIG. 2.

Alternatively, the fan-mounted air freshener device may comprise replaceable peel-n-stick adhesive-backed air-freshener packets. These packets can easily be affixed onto the surface of a fan's blades 11 (in the same way as the above noted conduits having a connection means). As noted, these air-freshener packets are disposable and can be purchased in multiunit packages. Such packets would necessarily be sealed prior to use, so that the air-freshener or deodorizer contained therein does not escape prematurely. This embodiment would provide air-freshening qualities for a more limited time period.

In use, the fan-mounted air freshener device 10 would be simple and straightforward to use. The user may set the scent-release to occur at desired time intervals. The user may choose from among a wide variety of scents and aromas, changing these as he or she pleased. For example, in summer the scent of mountain rain or jasmine may be pleasing; in winter, the scent of a pine forest and so forth.

There has thus been outlined, rather broadly, the more important features of the invention in order that the detailed description thereof that follows may be better understood, and in order that the present contribution to the art may be better appreciated.

Having thus described the invention in rather full detail, it will be understood that such detail need not be strictly adhered to, but that additional changes and modifications may suggest themselves to one skilled in the art. In particular, with respect to the above description, it is to be realized that the optimum dimensional relationships for the parts of the present invention may include variations in size, materials, shape, form, function and manner of operation. It is accordingly intended that such changes and modifications fall within the scope of the invention as defined by the subjoined claims.

What is claimed is:

1. A fan-mounted air freshener device for providing users with an easy and convenient means of emitting bursts of air-freshening scents or deodorizing sprays into a room, comprising:
(a) an electric fan;
(b) said electric fan comprising fan-blades having conduits;
(c) said conduits terminating in the form of a nozzle;
(d) a control unit for enabling the user to determine when the air-freshening scent or deodorizing spray is to be released;
(e) said control unit being powered by a power source, such as a battery or AC outlet;
(f) a scent reservoir comprising at least one tank capable of holding the air-freshening scent or deodorizing spray until directed by the user (by way of operation of the control unit) to emit said scent or spray; and
(g) a canal for conveying said emission of air-freshening scent or deodorizing spray from the scent reservoir to the conduits of the fan-blades;
whereby said air-freshening scent or deodorizing spray is conveyed by way of the canal from the scent reservoir to the fan-blades, said scent or spray continues to flow through the conduits and is ultimately emitted into the ambient air by way of the nozzle, where the centrifugal forces created by the whirling fan-blades fling droplets of scent or spray into the room in a rotary array.

2. The fan-mounted air freshener device recited by claim 1, where said electric fan is a ceiling fan.

3. The fan-mounted air freshener device recited by claim 1, where said electric fan is a table fan.

4. The fan-mounted air freshener device recited by claim 1, where the control unit comprises a plurality of control knobs for selecting an air-freshening scent or deodorizing spray from a variety of options that are available.

5. The fan-mounted air fresher device recited by claim 1, where said control unit is a wall-mounted control panel.

6. The fan-mounted air freshener device recited by claim 1, further comprising a second control unit which is a remote control unit.

7. The fan-mounted air freshener device recited by claim 1, further comprising a timer that is capable of being programmed by said user so that the air-freshening scent or deodorizing spray is released at predetermined time intervals; said control panel further comprising a plurality of control knobs allowing the user to program said timer.

8. The fan-mounted air freshener device recited by claim 1, where said nozzles are positioned near the ends of the fan-blades.

9. A fan-mounted air freshener device that can be installed onto an electric fan in order to provide a user with an easy and convenient means of emitting bursts of air-freshening scents or deodorizing sprays into a room, comprising:
(a) conduits that terminate in the form of a nozzle;
(b) said conduits having a connection means on a portion of their outer surface, said connection means being appointed to attach said conduits to the fan-blades of said electric fan;
(c) a control unit that enables the user to determine when the air-freshening scent or deodorizing spray is to be released;
(d) said control unit being powered by a power source, such as a battery or AC outlet;
(e) a scent reservoir comprising at least one tank capable of holding the air-freshening scent or deodorizing spray until directed by the user (by way of operation of the control unit) to emit said scent or spray; and
(f) a canal for conveying said emission of air-freshening scent or deodorizing spray from the scent reservoir to the conduits;
whereby said air-freshening scent or deodorizing spray is conveyed by way of the canal from the scent reservoir to the conduits, said scent or spray continues to flow through the conduits and is ultimately emitted into the ambient air by way of the nozzle, where the centrifugal forces created by the whirling fan-blades flings droplets of scent or spray into the room in a rotary array.

10. The fan-mounted air freshener device recited by claim 9, where said connection means is a peel-n-stick adhesive-backed component.

11. The fan-mounted air freshener device recited by claim 9, where said connection means is a thin layer of an adhesive.

12. The fan-mounted air freshener device recited by claim 9, where said electric fan is a ceiling fan.

13. The fan-mounted air freshener device recited by claim 9, where said electric fan is a table fan.

14. The fan-mounted air freshener device recited by claim 9, where the control unit comprises a plurality of control knobs for selecting an air-freshening scent or deodorizing spray from a variety of options that are available.

15. The fan-mounted air fresher device recited by claim 9, where said control unit is a wall-mounted control panel.

16. The fan-mounted air freshener device recited by claim 9, further comprising a second control unit which is a remote control unit.

17. The fan-mounted air freshener device recited by claim 9, further comprising a timer that is capable of being programmed by said user so that the air-freshening scent or deodorizing spray is released at predetermined time intervals; said control panel further comprising a plurality of control knobs allowing the user to program said timer.

18. The fan-mounted air freshener device recited by claim 9, where said nozzles are positioned near the ends of the fan-blades.

19. A fan-mounted air freshener device that can be installed onto the blades of an electric fan in order to provide a user with an easy and convenient means of emitting air-freshening scents or deodorizing sprays into a room, comprising:
(a) a disposable packet having an air-freshening scent or deodorizing spray contained therein; and
(b) the back of said packet having a peel-n-stick adhesive-backed component;
whereby the user can affix one or more of said packets to the surfaces of the fan-blades, and the centrifugal forces created by the whirling fan-blades carry the air-freshening scent or deodorizing spray throughout the room.

20. A fan-mounted air freshener device as recited by claim 19 where said air-freshener packet is available for purchase in multiunit packages.
